(19) United States
(12) Patent Application Publication
(10) Pub. No.: US 2019/0066829 A1
(43) Pub. Date: Feb. 28, 2019

(54) System and Method for Populating and Processing Prescription Scripts

(71) Applicant: Te Lim, Hagerstown, MD (US)

(72) Inventor: Te Lim, Hagerstown, MD (US)

(21) Appl. No.: 16/110,233

(22) Filed: Aug. 23, 2018

Related U.S. Application Data
(60) Provisional application No. 62/549,335, filed on Aug. 23, 2017.

(51) Int. Cl. G16H 20/10

(52) U.S. Cl. CPC G16H 20/10

(57) ABSTRACT

A system and method for populating and processing prescription scripts to reduce human error and save time while filling prescriptions. A pharmacy e-pad and a local server are utilized by a pharmacy to receive prescription orders, process the prescription orders, and fill the prescription orders. The pharmacy e-pad utilizes a touchscreen, a processing unit, and in some embodiments a digital scanner. The touchscreen allows a pharmacist or technician to directly input information for filling a prescription, while the processing unit processes inputs and outputs through the pharmacy e-pad. Meanwhile, the digital scanner can be used to retrieve information from scannable codes. The local server provides a means for storing data, facilitating communication between the pharmacy e-pad and other pharmacy devices, and facilitating communication with other pharmacy or third-party servers.

The current application claims a priority to the U.S. Provisional Patent application Ser. No. 62/549,335 filed on Aug. 23, 2017.

### FIELD OF THE INVENTION

The present invention relates generally to the process of filling prescriptions. More specifically, the present invention provides a system and method for populating and processing prescription scripts to reduce human error and save time while filling prescriptions.

### BACKGROUND OF THE INVENTION

Pharmacies have been dependent on the prescription paper pads since the first pharmacy in the 19th century. It's time to join the 21st century and advance our pharmacies to current technology. In today's retail pharmacies, pharmacists are pulled in many directions, therefore, time is of an essence. Besides checking and verifying that every prescription is correct, they are called to take customers/staff questions, take calls from doctors/other pharmacies, counsel every new medication, do medication therapy management (MTM), and give vaccinations. How can one pharmacist handle that entire work load and expect to meet customer's expectation of 20 minutes or less for their prescriptions?

The present invention will help reduce one of the most time-consuming duties of a pharmacist. That is the processing of prescriptions received via phone from a doctor's office or transferred from another pharmacy. It is an aim of the present invention to reduce the stress level of a pharmacist and give more time back to the pharmacist to perform other tasks. Let's examine what is required for a prescription phone in. When a prescription for a controlled substance is phoned in, it is required by law that the patient's address, phone number, and date of birth are written on the prescription. It is also required that a doctor's address, Drug Enforcement Agency (DEA) number, National Provider Identifier Standard (NPI) number, and phone number be on the prescription. It is very time consuming and very tedious to copy all of this information by hand. Therefore it is an object of the present invention to communicate with a pharmacy's computer and utilize a patient database and prescriber database to fill in required fields of a prescription form. This will save time for either the pharmacist or pharmacy technician when processing a pharmaceutical order.

### BRIEF DESCRIPTION OF THE DRAWINGS

FIG. 1 is a diagram depicting communications between the pharmacy e-pad and other devices or medium.

FIG. 2 is a diagram depicting the pharmacy e-pad being used for controlled substance compliance.

FIG. 3 is a flowchart depicting the steps for automatically populating a prescription form with patient data, medication data, and prescriber data.

FIG. 4 is a flowchart thereof, further depicting steps for unlocking the pharmacy e-pad.

FIG. 5 is a flowchart thereof, further depicting steps for sending the prescription form to another electronic device.

FIG. 6 is a flowchart thereof, further depicting steps for transferring the pharmaceutical order to another pharmacy.

FIG. 7 is a flowchart thereof, further depicting steps for retrieving the patient data, the medication data, and the prescriber data from a quick fill code.

FIG. 8 is a flowchart thereof, further depicting step for entering a prescriber selection into the pharmacy e-pad.

FIG. 9 is a flowchart thereof, further depicting step for populating the patient data in a subsequent prescription form.

FIG. 10 is a flowchart thereof, further depicting steps for populating the prescriber data in a subsequent prescription form.

FIG. 11 is a flowchart thereof, further depicting steps for maintaining controlled substance compliance.

FIG. 12 is a flowchart thereof, further depicting steps for performing monthly inventory of controlled substances.

### DETAIL DESCRIPTIONS OF THE INVENTION

All illustrations of the drawings are for the purpose of describing selected versions of the present invention and are not intended to limit the scope of the present invention.

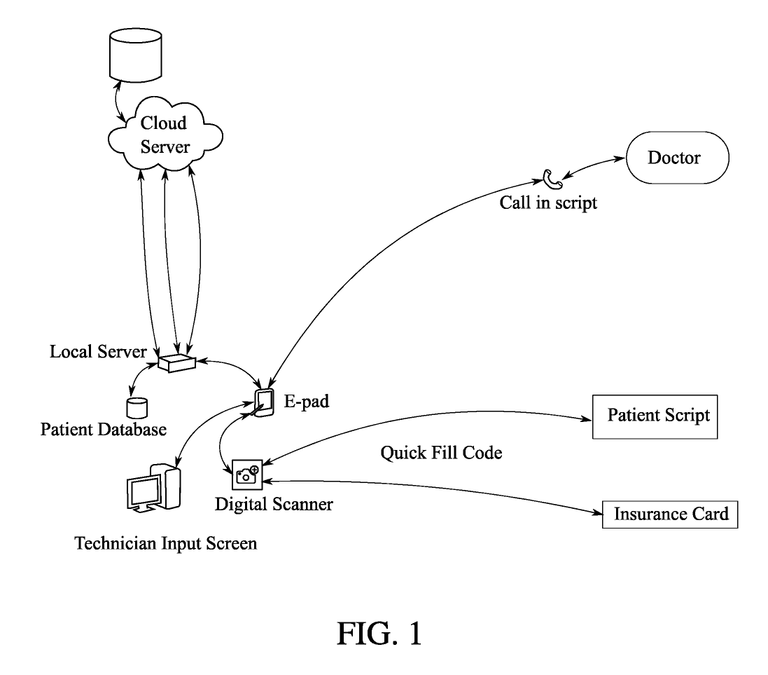

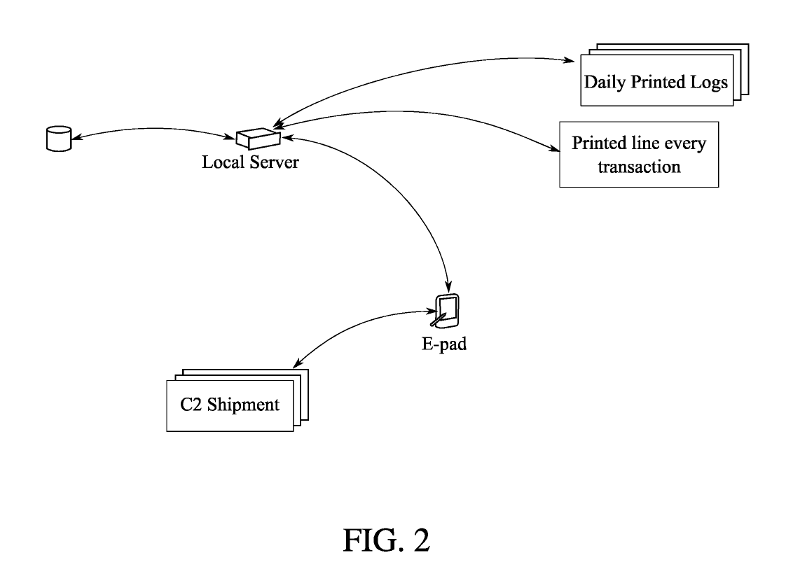

The present invention provides a system and method for populating and processing prescription scripts to reduce human error and save time while filling prescriptions. In reference to FIG. 1-2, a pharmacy e-pad and a local server are utilized by a pharmacy to receive prescription orders, process the prescription orders, and fill the prescription orders. The pharmacy e-pad comprises a touchscreen, a processing unit, and in some embodiments a digital scanner. The touchscreen allows a pharmacist or technician to directly input information for filling a prescription, while the processing unit processes inputs and outputs through the pharmacy e-pad. Meanwhile, the digital scanner can be used to retrieve information from scannable codes. The local server provides a means for storing data, facilitating communication between the pharmacy e-pad and other pharmacy devices, and facilitating communication with other pharmacy or third-party servers.

The pharmacy e-pad may be communicably coupled to the local server through a wired connection or through a wireless connection. As such, the pharmacy e-pad may include a port for connecting a cable or the cable may be hardwired in order to provide a wired connection to the local server. In order to provide a wireless connection with the local server, the pharmacy e-pad may comprise a transceiver, wherein the transceiver is electronically connected to the processing unit.

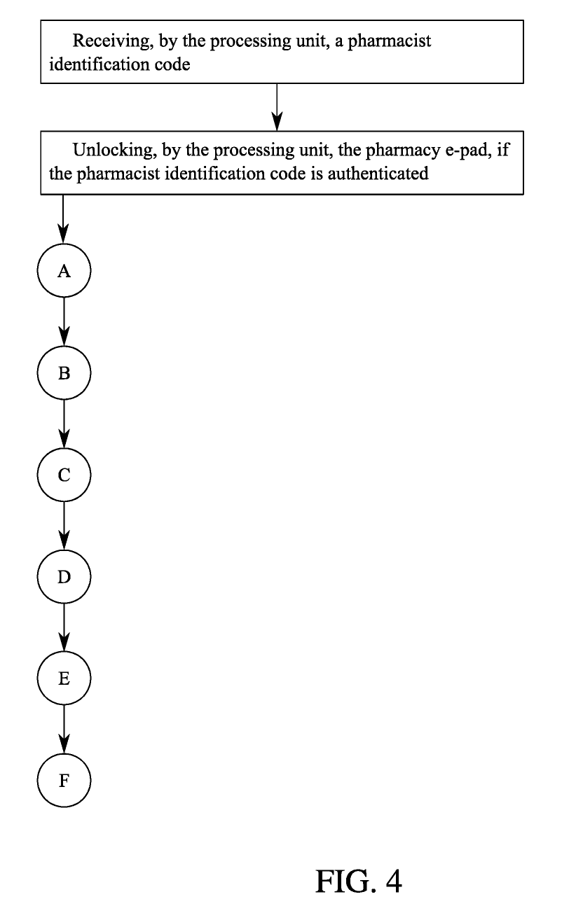

In reference to FIG. 4, to use the pharmacy e-pad, a pharmacist must first sign into a pharmacist account. To sign into the pharmacist account, the software application directs the pharmacist to enter a pharmacist identification code. The pharmacist identification code includes at least one unique identifier that is associated with the pharmacist account. In some embodiments, the pharmacist identification code may be a four pin identification code. In other embodiments, the pharmacist identification code may be a username and password. To gain full access to the pharmacist account, the pharmacist enters the pharmacist identification code through the touchscreen, wherein the processing unit receives the pharmacist identification code and proceeds to verify the pharmacist identification code.

If the processing unit is unable to authenticate the pharmacist identification code, then access to the pharmacist account is denied. The pharmacist may then attempt to re-enter the pharmacist identification code in order to gain access to the pharmacist account. In some embodiments, the software application may restrict the number of login attempts made by an individual. When the individual exceeds the number of login attempts, the software application may lock the pharmacist account or otherwise restrict further access to the pharmacist account. For example, the software application may institute a timed lockout, wherein the individual may not attempt to log in again for a predetermined length of time. In other embodiments, the software application may require the individual to submit alternative identification means or require an account reset from another party such as an administrator.

If the processing unit is able to authenticate the pharmacist identification code, then the processing unit unlocks the pharmacy e-pad, providing the pharmacist full access to the pharmacist account. Depending on the embodiment, the pharmacist may remain logged in indefinitely or for a pre-determined login period. In embodiments where the pharmacist remains logged in indefinitely, the pharmacist must manually log out of the pharmacist account in order to restrict future access to the pharmacist account. In embodiments wherein the pharmacist is logged in for a pre-determined login period, the software application automatically restricts access to the pharmacist account after a pre-determined length of time, wherein the pharmacist must re-enter the pharmacist identification code to re-gain access to the pharmacist account.

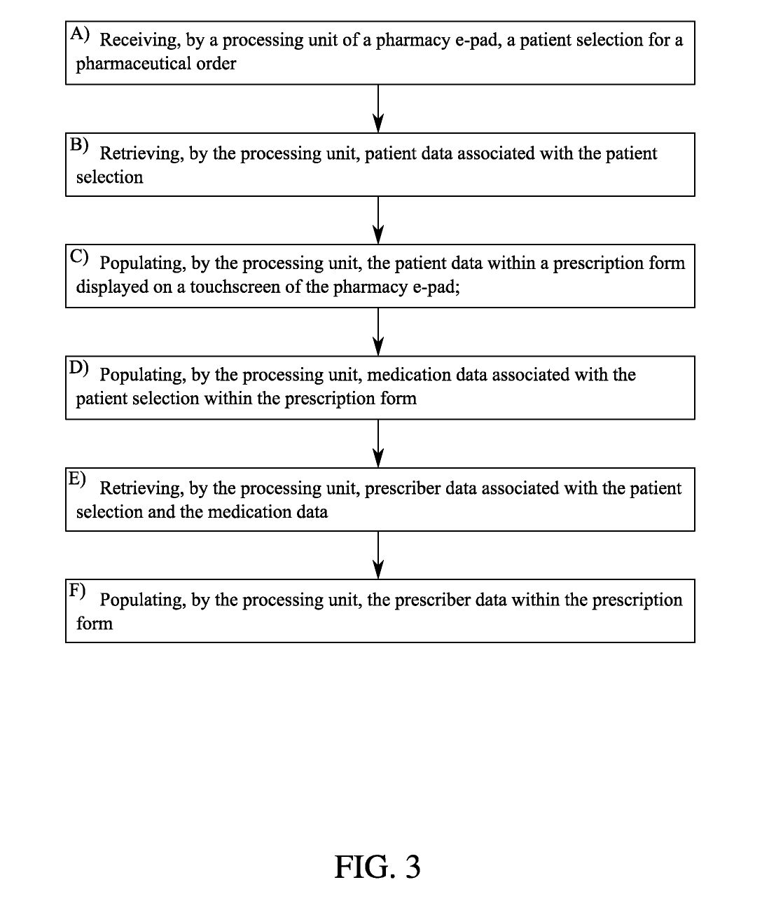

Once the pharmacist is logged into the pharmacist account, the pharmacist can begin processing a pharmaceutical order. In reference to FIG. 3, the processing unit first receives a patient selection for the pharmaceutical order. The patient selection directs the processing unit on which patient profile and associated info should be retrieved from a patient database. Preferably, the patient database is stored on the local server; however, the patient database may also be stored on the pharmacy e-pad or a remote server in other embodiments. Upon receiving the patient selection, the processing unit locates the patient profile within the patient database that is associated with the patient selection and retrieves patient data associated with the patient selection.

The patient selection may be received by the processing unit in multiple ways. In a first patient selection method, the patient selection is selected from a list of patients, wherein the list of patients is organized by date of birth. A date of birth field is accessible to the pharmacist via a prescription form displayed on the touchscreen, wherein the pharmacist may select the date of birth field and enter a birth date. Upon entering the birth date, the processing unit scans the patient database for all patients having the birth date and generates the list of patients. The pharmacist may then make the patient selection by clicking on the appropriate patient name from the list of patients. In reference to FIG. 3, once the pharmacist has entered the patient selection, the processing unit retrieves the patient data associated with the patient selection from the patient database.

In further reference to FIG. 3, the processing unit then populates the patient data within the prescription form displayed on the touchscreen. The patient data may include a patient name, a patient address, a patient phone number, a patient insurance number, etc. Each piece of information from the patient data is entered into a corresponding field of the prescription form. For example, the patient name is entered into a name field, the patient address is entered into an address field, the patient phone number is entered into a phone number field, the patient insurance number is entered into an insurance field, etc. In other embodiments, the pharmacist may fill in a data field other than the date of birth field, wherein the patient list is organized according to the corresponding data field. By automatically filling in the patient data based on the selection of a single data field, the pharmacist does not have to manually enter more than one piece of information, thus saving the pharmacist time and reducing the chance of human error by incorrectly filling in another data field.

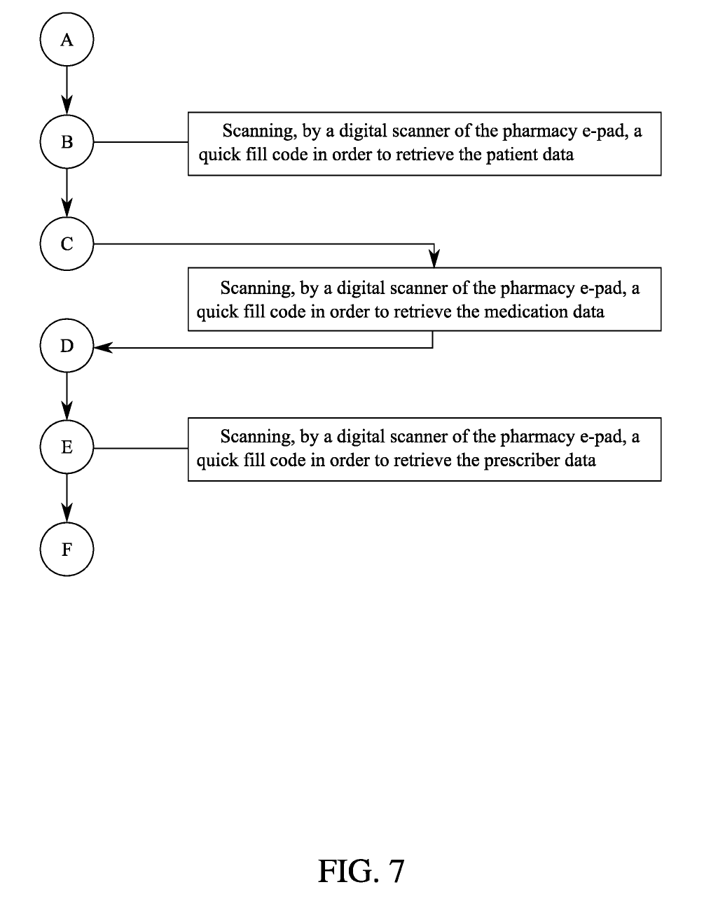

In reference to FIG. 7, in a second patient selection method, the patient selection is received through a quick fill code that is scanned by the digital scanner. In turn, the quick fill code is scanned by the digital scanner in order to retrieve the patient data. In some embodiments, the quick fill code is a quick response code, barcode, or other type of two-dimensional encoded image. In other embodiments, the quick fill code may be a chip, similar to that of a credit card. In yet other embodiments, the quick fill code may be a magnetic strip, similar to that of a credit card. As such, the digital scanner may be configured to scan and read more than one type of medium. The digital scanner may be configured to scan a two-dimensional image, scan a chip, scan a magnetic strip, scan another type of encoded media, or a combination thereof.

The quick fill code provides a unique identifier that is directly associated with the patient, such that when the quick fill code is scanned, the processing unit is able to determine the patient selection from the unique identifier and retrieve the patient data associated with the patient selection. In some embodiments, the unique identifier may be a Bank Identification Number (BIN), a Processor Control Number (PCN), a Member Identification Number, a Group Number, or a combination thereof. The processing unit then populates the patient data within the prescription form displayed on the touchscreen. In some embodiments, the patient data may be encoded directly within the quick fill code, wherein the processing unit retrieves the patient data directly from the quick fill code. The quick fill code may contain checksums to ensure data validity.

The quick fill code can be implemented with a number of different media. For example, in some instances the quick fill code may be placed on a printed prescription sheet or an insurance card. The quick fill code can be printed on, adhered to, or otherwise positioned on the printed prescription sheet or the insurance card, such that the quick fill code may be readily scanned by the digital scanner. In other embodiments, the quick fill code may be embedded in the insurance card, such as when the quick fill code is a chip, wherein the portion of the insurance code containing the quick fill code is inserted into the digital scanner in order to scan the quick fill code.

In reference to FIG. 3, once the patient data has been populated into the prescription form, medication data associated with the patient selection can be populated into the prescription form by the processing unit. The medication data may be received by the processing unit in multiple ways. In a first medication data retrieval method, the medication data is received by the processing unit through the touchscreen, wherein a free writing field is provided on the prescription form. The pharmacist is able to write directly into the free writing field through the touchscreen, wherein the medication data is received by the processing unit via the touchscreen and is displayed in real-time on the touchscreen. The pharmacist can use the free writing field to insert the name of the medication, the strength of the prescription, directions for taking the medication, the quantity of the medication, the number of prescribed refills, etc.

In reference to FIG. 7, in a second medication data retrieval method, the medication data is retrieved via the quick fill code. The medication data may be stored in the patient profile, wherein the processing unit is directed to retrieve the medication data from the patient profile in conjunction with the patient data, upon scanning the quick fill code. In other embodiments, the medication data may be encoded directly by the quick fill code, wherein the medication data is retrieved and decoded by the processing unit directly upon scanning the quick fill code with the digital scanner. The second medication data retrieval method is particularly useful for refilling prescriptions, as the medication data has already been entered for the initial prescription order and can be easily stored by the processing unit.

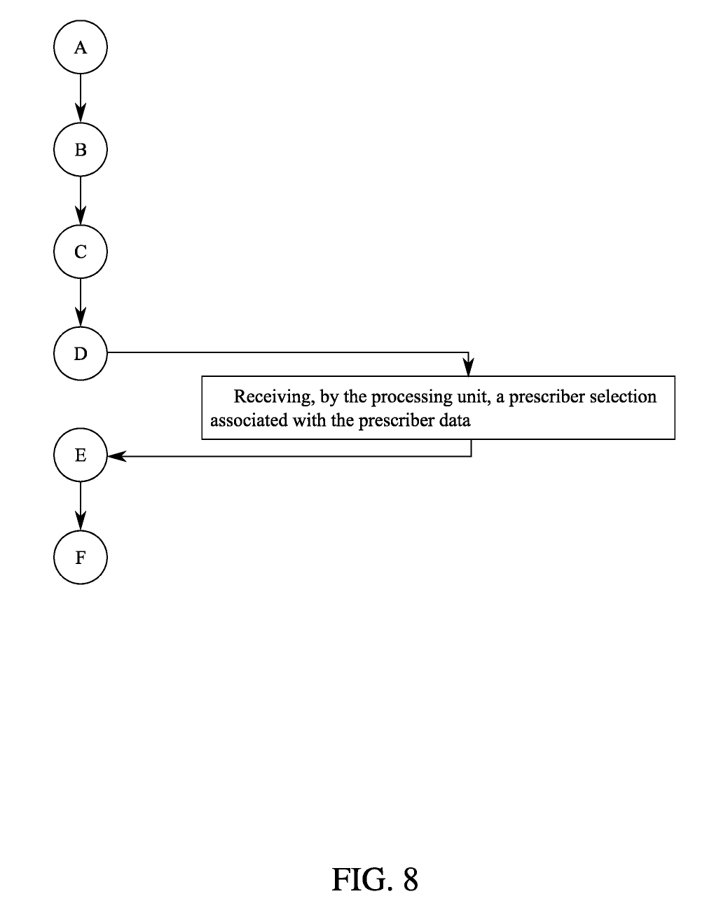

In reference to FIG. 3, once the patient data and the medication data has been populated into the prescription form, the processing unit retrieves prescriber data associated with the patient selection and the medication data. The prescriber data may be retrieved by the processing unit in multiple ways. In reference to FIG. 8, in a first prescriber selection method, the processing unit first receives a prescriber selection associated with the prescriber data, wherein the prescriber selection is selected from a list of prescribers. A prescriber phone number field, a Drug Enforcement Agency (DEA) number field, and a National Provider Identifier Standard (NPI) field is accessible to the pharmacist via the prescription form displayed on the touchscreen. The pharmacist may select any one of the prescriber phone number field, the DEA number field, or the NPI field. Upon selecting one of the three fields, the processing unit retrieves the list of prescribers from a prescriber database stored on the local server. The pharmacist may then make the prescriber selection by clicking on the appropriate prescriber from the list of prescribers. Once the pharmacist has entered the prescriber selection, the processing unit retrieves the prescriber data. The processing unit then populates the prescriber data within the prescription form.

In reference to FIG. 7, in a second prescriber selection method, the prescriber data is retrieved via the quick fill code. The prescriber data may be stored in the patient profile, wherein the processing unit is directed to retrieve the prescriber data from the patient profile in conjunction with the patient data and the medication data, upon scanning the quick fill code. In other embodiments, the prescriber data may be encoded directly by the quick fill code, wherein the prescriber data is retrieved and decoded by the processing unit directly upon scanning the quick fill code with the digital scanner. The second prescriber selection method is particularly useful for refilling prescriptions, as the prescriber data has already been entered for the initial prescription order and can be easily stored by the processing unit.

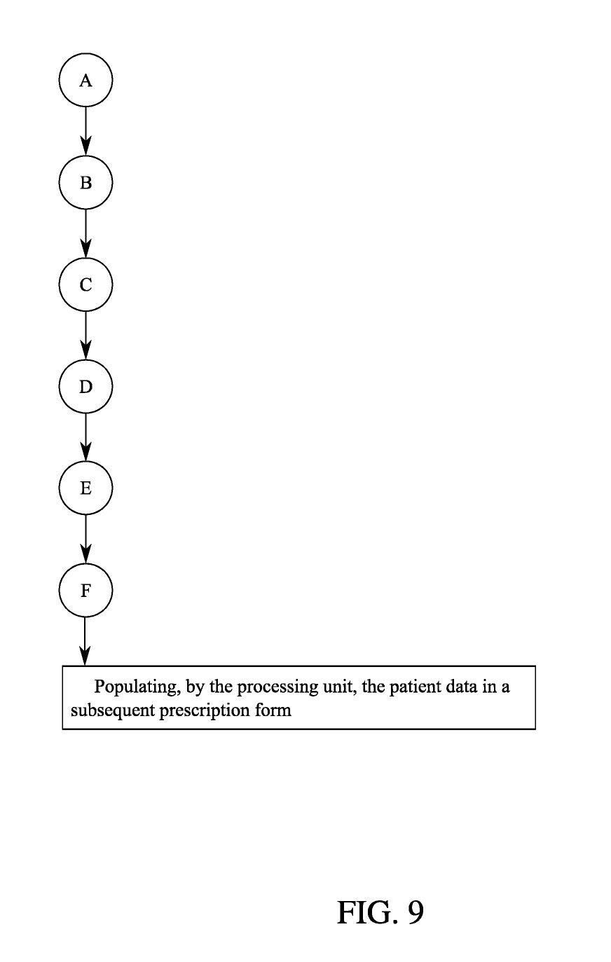

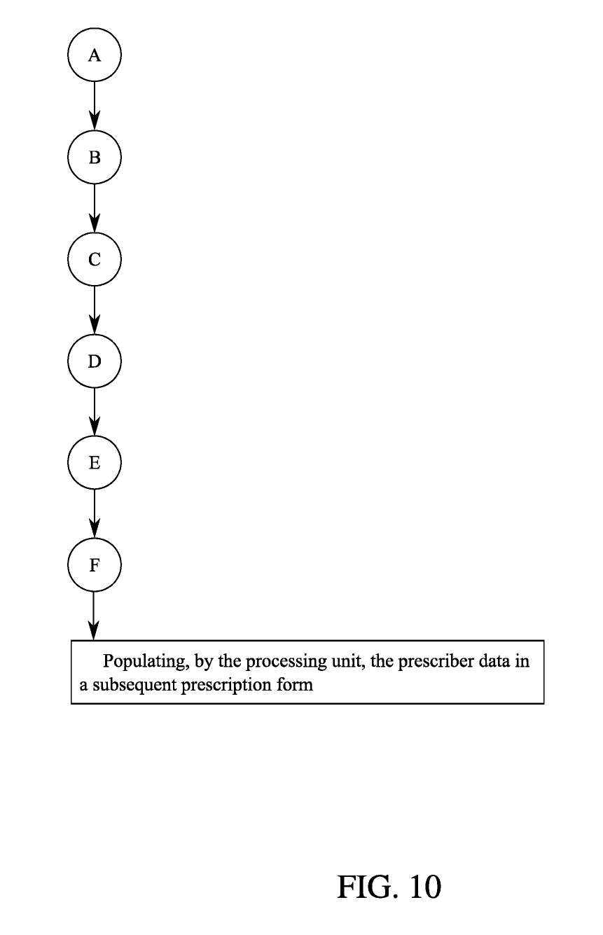

In some instances, the pharmacist may need to process a subsequent prescription form, wherein the patient data and/or the prescriber data is the same. If the subsequent prescription form is for the same patient, then the pharmacist will indicate as so through the software application. The processing unit will then populate the patient data and the prescriber data into the subsequent prescription form, as depicted in FIG. 9. New medication data can then be filled in in order to complete the subsequent prescription form. If the subsequent prescription form is for a different patient but from the same prescriber, then the pharmacist will indicate as so through the software application. The processing unit will then populate the prescriber data into the subsequent prescription form, as depicted in FIG. 10. New patient data and new medication data can then be filled in in order to complete the subsequent prescription form.

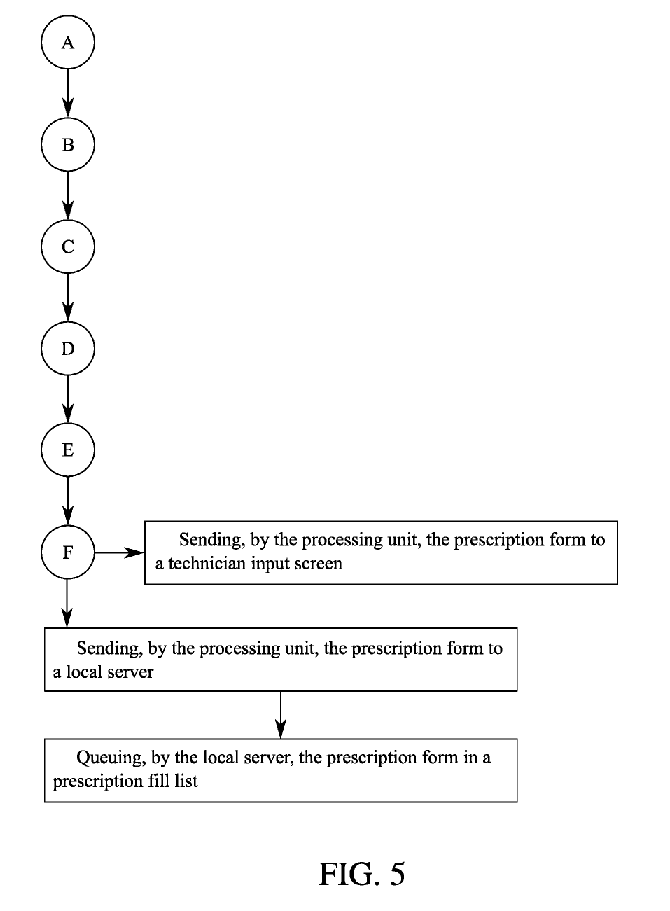

Once the patient data, the medication data, and the prescriber data have been populated into the prescription form, the pharmaceutical order can be processed. In reference to FIG. 5, the processing unit may send the prescription form to the local server, wherein the local server queues the prescription form in a prescription fill list. The prescription form will then be displayed on a technician input screen when the prescription form is moved up to the top of the prescription fill list. In some embodiments, the prescription form may be sent directly from the processing unit to the technician input screen, bypassing the prescription fill list.

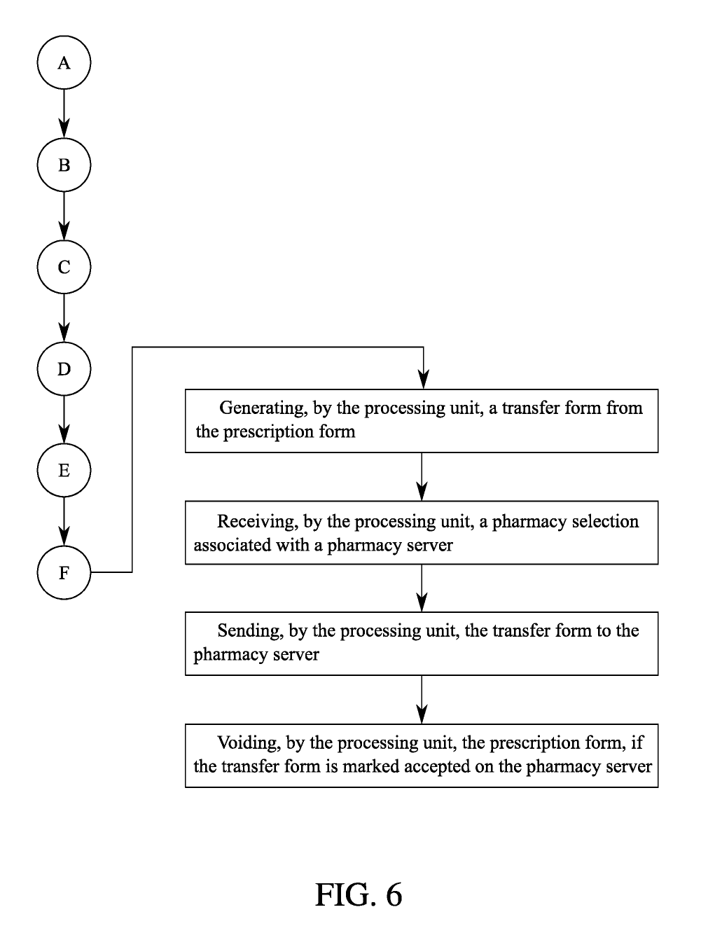

The present invention also allows for the quick and easy transfer prescriptions from one pharmacy to another. In reference to FIG. 6, a prescription transfer is initiated by the pharmacist through the software application, wherein the processing unit generates a transfer form from the prescription form. The transfer form may include some or all of the patient data, the medication data, and the prescriber data. Additionally, the transfer form may include the name of the pharmacist and information about the pharmacy that is initiating the prescription transfer, such as the pharmacy name, pharmacy address, pharmacy phone number, and pharmacy DEA number. The pharmacist must also input a pharmacy selection for the pharmacy receiving the transfer form, wherein the pharmacy selection is associated with a pharmacy server of the pharmacy receiving the transfer form.

In some embodiments, the pharmacy selection is selected from a list of pharmacies. A pharmacy selection field is accessible to the pharmacist via the transfer form displayed on the touchscreen, wherein the pharmacist may select the pharmacy selection field to display the list of pharmacies. The pharmacist may then make the pharmacy selection by clicking on the appropriate pharmacy name from the list of pharmacies. Once the pharmacist has entered the pharmacy selection, the processing unit sends the transfer form to the pharmacy server. In some embodiments, the transfer form is generated by the pharmacy e-pad and then sent to the local server, wherein the local server sends the transfer form to a cloud server which in turn sends the transfer form to the pharmacy server.

Once the transfer form is received by the pharmacy server, a notification is generated to alert a subsequent pharmacist of the pending status of the transfer form. The subsequent pharmacist may then review the transfer form on a subsequent e-pad. If the subsequent pharmacist accepts the transfer form, then a new prescription form is generated on the subsequent e-pad using the patient data, the medication data, and the prescriber data on the transfer form. Additionally, a notification is sent to the initial pharmacy, wherein the transfer form is marked as accepted. The processing unit then voids the prescription form if the transfer form is marked as accepted on the pharmacy server, such that the prescription form is no longer fillable through the initial pharmacy, as depicted in FIG. 6.

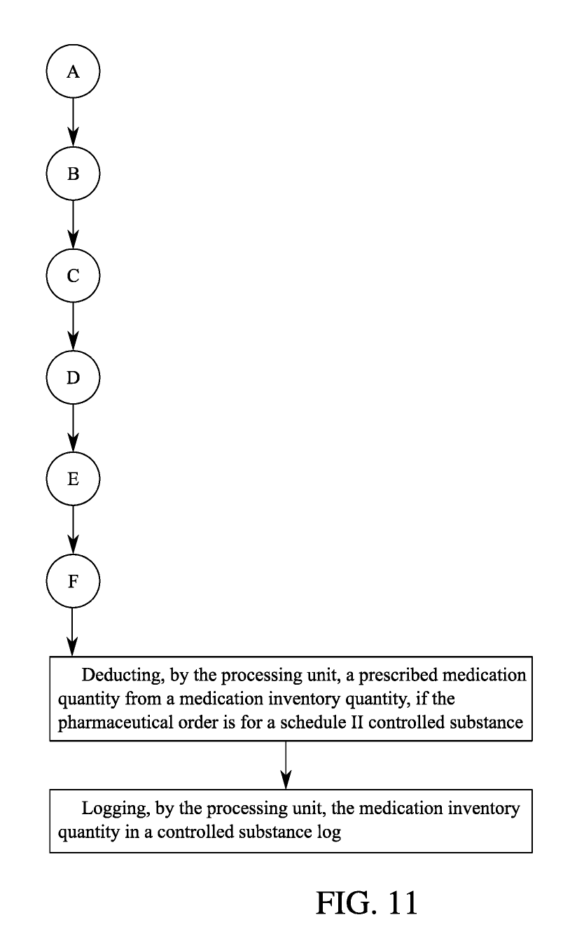

The present invention can also be used to maintain compliance when handling controlled substances. When the pharmacy receives a schedule II controlled substance (C2) narcotic invoice, a quick response code or other similar scannable code will be on the C2 narcotic invoice. The pharmacist can then scan the quick response code with the digital scanner of the pharmacy e-pad, wherein narcotic information for the C2, such as the type of narcotic and the quantity of the narcotic, is retrieved by the processing unit and stored on the local server. Subsequently and in reference to FIG. 11, if the pharmaceutical order being processed is for a C2, then the processing unit deducts a prescribed medication quantity from a medication inventory quantity. The processing unit then logs the medication inventory quantity in a controlled substance log.

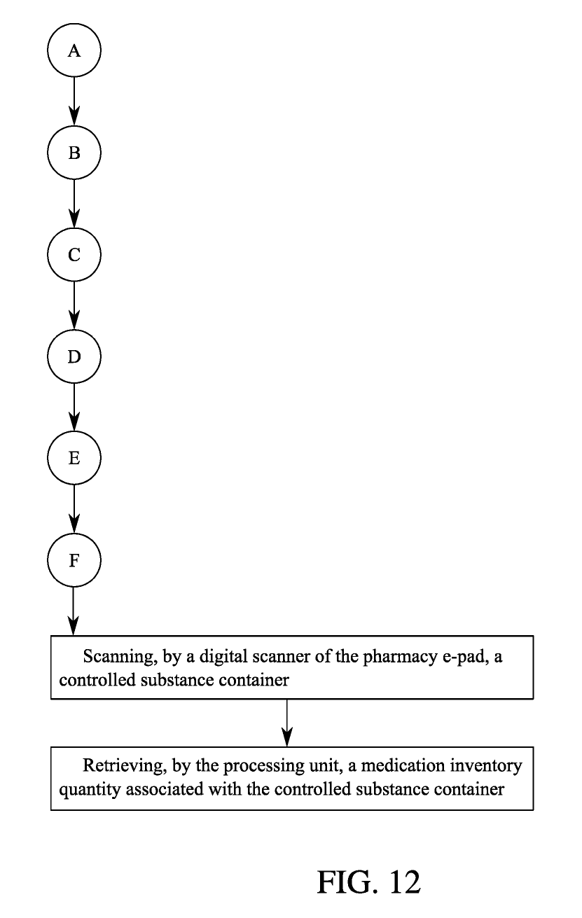

The pharmacy e-pad can also be used to perform monthly inventory of C2s. In reference to FIG. 12, the pharmacist can use the digital scanner to scan a controlled substance container, wherein the processing unit then retrieves the medication inventory quantity associated with the controlled substance container. The medication inventory quantity is displayed to the pharmacist through the touchscreen and the pharmacist is prompted to enter a physical inventory count. The pharmacist may then proceed to count the physical inventory within the controlled substance container and enter the physical inventory count through the touchscreen. The processing unit then compares the physical inventory count to the medication inventory quantity. If the physical inventory count does not match the medication inventory quantity, then the pharmacy e-pad will ask the pharmacist to submit a reason for the disparity. The pharmacist may then proceed with following company policy on reporting a shortage or overage to the DEA.

The controlled substance log and monthly inventory reports can be printed to provide a physical copy for compliance records. In some embodiments, the pharmacy e-pad may be connected to a printer through a wired or wireless connection in order to print the controlled substance log and the monthly inventory reports. In other embodiments, the printer may be integrated with the pharmacy e-pad, wherein the controlled substance log and the monthly inventory reports can be printed directly from the pharmacy e-pad. The printer can also be used to print an image of the prescription form with a barcode and end of day label for record keeping and filing with other prescription records.

Although the invention has been explained in relation to its preferred embodiment, it is to be understood that many other possible modifications and variations can be made without departing from the spirit and scope of the invention as hereinafter claimed.

What is claimed is:

1. A method for populating and processing prescription scripts by executing computer-executable instructions stored on a non-transitory computer readable medium, the method comprises the steps of:
receiving, by a processing unit of a pharmacy e-pad, a patient selection for a pharmaceutical order;
retrieving, by the processing unit, patient data associated with the patient selection;
populating, by the processing unit, the patient data within a prescription form displayed on a touchscreen of the pharmacy e-pad;
populating, by the processing unit, medication data associated with the patient selection within the prescription form;
retrieving, by the processing unit, prescriber data associated with the patient selection and the medication data; and
populating, by the processing unit, the prescriber data within the prescription form.

2. The method for populating and processing prescription scripts by executing computer-executable instructions stored on a non-transitory computer readable medium, the method as claimed in claim 1 further comprises the step of:
sending, by the processing unit, the prescription form to a technician input screen.

3. The method for populating and processing prescription scripts by executing computer-executable instructions stored on a non-transitory computer readable medium, the method as claimed in claim 1 further comprises the step of:
sending, by the processing unit, the prescription form to a local server.

4. The method for populating and processing prescription scripts by executing computer-executable instructions stored on a non-transitory computer readable medium, the method as claimed in claim 3 further comprises the step of:
queuing, by the local server, the prescription form in a prescription fill list.

5. The method for populating and processing prescription scripts by executing computer-executable instructions stored on a non-transitory computer readable medium, the method as claimed in claim 1 further comprises the steps of:
generating, by the processing unit, a transfer form from the prescription form;
receiving, by the processing unit, a pharmacy selection associated with a pharmacy server; and
sending, by the processing unit, the transfer form to the pharmacy server.

6. The method for populating and processing prescription scripts by executing computer-executable instructions stored on a non-transitory computer readable medium, the method as claimed in claim 5 further comprises the step of:
voiding, by the processing unit, the prescription form, if the transfer form is marked accepted on the pharmacy server.

7. The method for populating and processing prescription scripts by executing computer-executable instructions stored on a non-transitory computer readable medium, the method as claimed in claim 1, wherein the patient selection is selected from a list of patients.

8. The method for populating and processing prescription scripts by executing computer-executable instructions stored on a non-transitory computer readable medium, the method as claimed in claim 7, wherein the list of patients is organized by date of birth.

9. The method for populating and processing prescription scripts by executing computer-executable instructions stored on a non-transitory computer readable medium, the method as claimed in claim 1, wherein the medication data is received by the processing unit via the touchscreen.

10. The method for populating and processing prescription scripts by executing computer-executable instructions stored on a non-transitory computer readable medium, the method as claimed in claim 1 further comprises the step of:
scanning, by a digital scanner of the pharmacy e-pad, a quick fill code in order to retrieve the patient data.

11. The method for populating and processing prescription scripts by executing computer-executable instructions stored on a non-transitory computer readable medium, the method as claimed in claim 1 further comprises the step of:
scanning, by a digital scanner of the pharmacy e-pad, a quick fill code in order to retrieve the medication data.

12. The method for populating and processing prescription scripts by executing computer-executable instructions stored on a non-transitory computer readable medium, the method as claimed in claim 1 further comprises the step of:
scanning, by a digital scanner of the pharmacy e-pad, a quick fill code in order to retrieve the prescriber data.

13. The method for populating and processing prescription scripts by executing computer-executable instructions stored on a non-transitory computer readable medium, the method as claimed in claim 1 further comprises the step of:
receiving, by the processing unit, a prescriber selection associated with the prescriber data.

14. The method for populating and processing prescription scripts by executing computer-executable instructions stored on a non-transitory computer readable medium, the method as claimed in claim 13, wherein the prescriber selection is selected from a list of prescribers.

15. The method for populating and processing prescription scripts by executing computer-executable instructions stored on a non-transitory computer readable medium, the method as claimed in claim 1 further comprises the step of:
populating, by the processing unit, the patient data in a subsequent prescription form.

16. The method for populating and processing prescription scripts by executing computer-executable instructions stored on a non-transitory computer readable medium, the method as claimed in claim 1 further comprises the step of:
populating, by the processing unit, the prescriber data in a subsequent prescription form.

17. The method for populating and processing prescription scripts by executing computer-executable instructions stored on a non-transitory computer readable medium, the method as claimed in claim 1 further comprises the steps of:
deducting, by the processing unit, a prescribed medication quantity from a medication inventory quantity, if the pharmaceutical order is for a schedule II controlled substance; and
logging, by the processing unit, the medication inventory quantity in a controlled substance log.

18. The method for populating and processing prescription scripts by executing computer-executable instructions stored on a non-transitory computer readable medium, the method as claimed in claim 1 further comprises the steps of:
scanning, by a digital scanner of the pharmacy e-pad, a controlled substance container; and
retrieving, by the processing unit, a medication inventory quantity associated with the controlled substance container.

19. The method for populating and processing prescription scripts by executing computer-executable instructions stored on a non-transitory computer readable medium, the method as claimed in claim 1 further comprises the steps of:
receiving, by the processing unit, a pharmacist identification code; and
unlocking, by the processing unit, the pharmacy e-pad, if the pharmacist identification code is authenticated.
